(19) United States
(12) Patent Application Publication
(10) Pub. No.: US 2019/0374119 A1
(43) Pub. Date: Dec. 12, 2019

(54) METHOD FOR DETERMINING THE MECHANICAL PROPERTIES OF A PELVIC CAVITY, AND MEASURING DEVICE

(71) Applicants: UNIVERSITE DE LILLE, LILLE (FR); ECOLE CENTRALE DE LILLE, VILLENEUVE D'ASCQ (FR); CENTRE NATIONAL DE LA RECHERCHE SCIENTIFIQUE, PARIS (FR); CENTRE HOSPITALIER REGIONAL UNIVERSITAIRE DE LILLE, LILLE (FR); ECOLE NATIONALE SUPERIEURE D?ARTS ET METIERS, PARIS (FR)

(72) Inventors: Mathias BRIEU, Villeneuve D'Ascq (FR); Michel COSSON, Lambersart (FR); Chrystèle RUBOD DIT GUILLET, Marcq En Baroeul (FR); Pauline LECOMTE, Faches Thumesnil (FR); Jean-François WITZ, Hellemmes (FR); Laurent PATROUIX, Mouvaux (FR)

(73) Assignees: UNIVERSITE DE LILLE, LILLE (FR); ECOLE CENTRALE DE LILLE, VILLENEUVE D'ASCQ (FR); CENTRE NATIONAL DE LA RECHERCHE SCIENTIFIQUE, PARIS (FR); CENTRE HOSPITALIER REGIONAL UNIVERSITAIRE DE LILLE, LILLE (FR); ECOLE NATIONALE SUPERIEURE D?ARTS ET METIERS, PARIS (FR)

(21) Appl. No.: 16/487,225

(22) PCT Filed: Feb. 20, 2018
(86) PCT No.: PCT/FR2018/050394

§ 371 Date: Aug. 20, 2019

(30) Foreign Application Priority Data
Feb. 21, 2017 (FR) 1751369

(51) Int. Cl. A61B 5/03; A61B 5/00; A61B 5/107

(52) U.S. Cl. CPC A61B 5/036; A61B 5/4337; A61B 5/1076; A61B 5/055; A61B 2562/0247; A61B 5/4325; A61B 5/6847

(57) ABSTRACT

The present invention provides a method (30) of determining mechanical properties of the pelvic cavity of a person or an animal, the pelvic cavity including a plurality of organs and the method comprising a step (34) during which pressure is measured at one or more points of the surface of one of the organs of said pelvic cavity, and during which, simultaneously, movements of a plurality of organs of said pelvic cavity are also measured.

The present invention also provides a measuring device for measuring pressure in an organ of the pelvic cavity in order to perform the above method (30). The measuring device comprises an optical fiber pressure sensor mounted in a non-metallic housing, and a closed flexible reservoir mounted in said non-metallic housing and having a surface, in particular a flexible surface, that constitutes a pressure measuring surface.

### BACKGROUND OF THE INVENTION

The present invention relates to determining the mechanical behavior of a portion of the human body. In particular, the present invention relates to determining the mechanical behavior of a person's pelvic cavity. The present invention also relates to a measuring device for making such a determination.

The pelvic cavity of woman is made up of pelvic organs, in particular the vagina, the bladder, the rectum, and the uterus. The pelvic organs are connected to one another and to bony portions by ligaments and by fasciae, and they are supported by the pelvic floor. The pelvic floor is the set of pelvic floor muscles that create an equilibrium referred to as “pelvic posture”, and that allows the pelvic organs of woman to have the physiological mobilities they need to perform their functions.

Pelvic organs have physiological mobilities that are quite large. Nevertheless, there exist common disorders of the pelvic posture of woman that affects these mobilities: for example, endometriosis leads to hypo-mobility, or on the contrary, genital prolapse leads to hyper-mobility of the pelvic organs.

The vagina is a cavity closely involved in maintaining the pelvic system of woman since it is situated between the bladder and the rectum, and numerous ligaments that play an important role in the pelvic posture are connected to the vaginal cuff or the vicinity of the cervix of the uterus. The magnitude of the stresses to which this organ is subjected (intra-abdominal pressure, gravity, weight of the viscera, coughing, . . . ) are all forces leading to all of the organs moving as a result of the rigidity of the tissues. Nevertheless, tissue rigidities are nowadays still evaluated poorly and in generic manner.

It is known to make use of devices, such as intra-vaginal probes, for performing measurements in vivo inside a patient's vaginal cavity. By way of example, such measurements may be measurements of intra-vaginal pressure during stress testing. Such devices thus make it possible to know pressure values that are specific to the person in question, while performing various stress exercises, so as to obtain a better understanding of potential disorders in the patient's pelvic posture.

Nevertheless, such devices do not enable the mechanical properties of a person's pelvic tissues to be characterized in a manner that is not invasive and not destructive. However, being able to determine the mechanical properties specific to the pelvic tissues of a particular woman would make it possible to obtain better evaluations of pathologies such as prolapse, or of risks, e.g. prior to childbirth. This would also make it possible to obtain significant therapeutic improvements such as targeting failing tissues, proposing personalized therapeutic strategies, or defining surgical prostheses that are better tolerated by the person since they are well adapted to that person's failing zones.

It is also known to construct a behavior model based on the histological composition of tissues so as to enable their hyperelastic nature, aging, or indeed a pathology to be modeled on the basis of a single parameter. Nevertheless, all of the data is obtained by destructive characterization on tissues that have been taken from a patient or from a cadaver, and at present there is no way of characterization of pelvic tissues in vivo, i.e. of performing non-destructive characterization of pelvic tissues.

Likewise, it is also known to construct a digital model that is specific to a patient on the basis of magnetic resonance imaging (MRI). Nevertheless, once again, the mechanical data concerning tissue that is used in such a model is not data concerning a patient's own tissues, but generic values taken from the literature or obtained from tissues that have been taken from the patient or from a cadaver.

### OBJECT AND SUMMARY OF THE INVENTION

The present invention seeks to solve the various above-mentioned technical problems. In particular, the present invention seeks to propose a method, and the corresponding device, enabling the mechanical properties of a person's pelvic cavity to be determined in non-destructive manner, and in particular in vivo, specifically for diagnostic purposes and for applying therapy to pelvic pathologies that is better adapted to each patient.

Thus, in an aspect, there is provided a method of determining, in particular in non-destructive manner, mechanical properties of the pelvic cavity of a person or of an animal, the pelvic cavity including a plurality of organs. The method comprises a step during which pressure is measured at one or more points of the surface of one of the organs of said pelvic cavity, and during which, simultaneously, movements of a plurality of organs of said pelvic cavity are also measured.

Thus, by simultaneously measuring pressure at one or more points and also measuring movements of a plurality of organs, it becomes possible to characterize certain tissues in the person's pelvic cavity in mechanical terms, and consequently to obtain a model of said pelvic cavity that is accurate and specific to the person on whom the measurements were taken. It becomes possible to obtain better knowledge of the anatomy of the patient in question, and to understand malfunctions that are present or that might arise in the future.

Preferably, the method is performed in order to determine the mechanical properties of the pelvic cavity of a living person or animal.

In particular, intra-vaginal or intra-rectal pressure is measured during an MRI examination in order to take simultaneous measurements of pressure and of organ movements. MRI is a conventional tool for diagnosing pelvic pathologies and it enables pelvic anatomical structures to be observed while they are at rest by means of static MRI, or else while they are moving by means of dynamic MRI. Herein, the advantage is to measure simultaneously intra-vaginal or intra-rectal pressure under stress and also to observe the movements caused by that stress by means of MRI imaging. Observing the movements of organs coupled with quantification of the pressures being exerted makes it possible to obtain better diagnosis of disorders of pelvic posture. Furthermore, knowing simultaneously the loading and the mobilities that are induced also makes it possible in vivo to characterize indirectly the mechanical properties of the patient's tissues (organs, ligaments and muscles involved in pelvic posture), thus making it possible to understand pelvic pathologies and to improve their diagnosis and treatment.

Preferably, said organ on the surface of which pressure is measured is the vagina or the rectum. The method then serves to evaluate the characteristics of certain particular organs, the vagina or the rectum, thus also making it possible to use a probe for taking local pressure measurements.

Preferably, the movements of said pelvic cavity are measured from data obtained by MRI, e.g. data obtained by dynamic MRI of the person or of the animal. The movements are determined globally, i.e. at a multitude of points in the pelvic cavity. Dynamic MRI serves in particular to observe accurately and to measure the movements of various organs in the patient's pelvic cavity. This makes it possible to obtain movements that are specific to the patient, thereby making it possible in the end to obtain characterization of the patient's pelvic cavity that is reliable.

Preferably, the method also includes a step of constructing a digital model of the pelvic cavity from imaging data of the shape of the pelvic cavity, e.g. from data obtained by static MRI of the person or of the animal, and optionally from standard mechanical properties. In this implementation, the digital model is constructed from anatomical data of the patient, thus making it possible for the digital model to have a shape that corresponds exactly to the patient's anatomy.

Preferably, construction of the digital model includes subdividing the digital model into finite elements. This is a conventional technique for constructing a digital model, and it serves to limit the amount of calculation needed while obtaining an appropriate model of the cavity.

In an implementation, the mechanical properties used in the digital model are modified in such a manner that the movements obtained with the digital model of said plurality of organs approach the movements as measured when the pressures at said one or more points of the surface of one of the organs of the digital model are equal to the measured pressures. The simultaneous measurements are thus used to refine the digital model constructed from the static MRI data: by comparing the movements obtained firstly from the digital model and secondly from the person, it is possible to modify the parameters of the digital model in order to minimize differences between the movements calculated from the digital model and the measured movements of the pelvic cavity. Parameters of the digital model are thus modified by image correlation, for a given pressure, between parameters provided by the digital model and parameters obtained by MRI. Mechanical properties are thus identified by an inverse method that consists in determining the mechanical properties that serve to minimize differences between the values obtained by the digital model and the values measured on the person.

In an implementation, the method also includes, after modifying the mechanical properties of the digital model, a step of modifying the digital model, e.g. modifying its shape or modifying a mechanical property, in order to simulate possible mechanical behavior of the pelvic cavity of the person or of the animal. Such a step of the method is performed when the digital model is considered as being a correct representation of the patient's pelvic cavity: it then becomes possible in the digital model to simulate operations that are being envisaged in order to verify that the behavior of the pelvic cavity, after the operation, will indeed be as expected. It is thus possible to perform prevention or diagnosis by making use solely of the digital model.

In another aspect, there is also provided a measuring device for measuring pressure in an organ of the pelvic cavity. The device comprises at least an optical fiber pressure sensor mounted in a non-metallic housing, and a closed flexible reservoir mounted in said non-metallic housing and having a surface, in particular a flexible surface, that constitutes a pressure measuring surface. The pressure measuring surface is for putting into contact with a surface of the organ of the cavity and the flexible reservoir is configured to transmit pressure exerted on the measuring surface to the optical fiber sensor.

Such a device presents the advantage of enabling pressure to be measured without requiring the use of metal elements. Specifically, performing magnetic resonance imaging (MRI) generates a magnetic field that is strong, which means it is not possible to insert any magnetic, ferrous, or conductive material, and thus as a general rule hardly any metal material. In addition, all existing technologies used for intra-vaginal pressure measurements require electrical signals to be transmitted in order to acquire data. Unfortunately, electrical signals are likely to be greatly disturbed by the presence of magnetic fields.

The flexible reservoir is closed so that it always contains the same quantity of fluid. Thus, the flexible reservoir is not intended to change volume, in particular by inflation, once it is made to bear against walls by exerting pressure thereon. The flexible reservoir always contains the same quantity of fluid and it is positioned inside a non-metallic housing so as to leave accessible only one surface, the measuring surface.

Preferably, the flexible reservoir comprises a flexible or deformable material that may be elastic or non-elastic. In particular, the flexible reservoir may be made of flexible or deformable material that is elastic or of flexible or deformable material that is not elastic. Thus, the flexible reservoir can deform under the effect of stress exerted thereon, but will not increase or decrease in volume, as would happen in particular with a reservoir that is capable of being inflated.

Thus, since the flexible reservoir is not to deform in order to bear against the wall to be measured, it does not deform the cavity in which it is used.

The flexible reservoir may be formed by a closed peripheral flexible membrane arranged inside the non-metallic housing: the non-metallic housing then includes an opening or window through which a portion of the membrane is accessible, i.e. the measuring surface. In such an embodiment, any pressure variation exerted on the measuring surface is transmitted to the remainder of the membrane of the flexible reservoir. Alternatively, the flexible reservoir may be formed firstly by the inside surface of the non-metallic housing, which includes an opening, and secondly by a flexible membrane that closes said opening of the non-metallic housing in order to form the measuring surface. In this embodiment, any pressure variation exerted on the measuring surface gives rise to a modification to the pressure inside the reservoir.

Either way, it can be understood that only a portion of the flexible reservoir, i.e. the measuring surface, becomes deformed under the stress exerted by the wall of the cavity, and that the remainder of the reservoir is protected by the non-metallic housing and is not subjected to any stress.

Finally, the size of the measuring surface depends only on the size of the opening in the housing: it is thus possible to make a local pressure measurement through the opening of the non-metallic housing without taking account of pressures exerted around the measuring surface.

The use of optical fibers makes it possible to take the pressure measurements while using materials that are compatible with an MRI environment. Specifically, optical fibers are non-metallic, and simultaneously the light signal representing the measured pressure value is insensitive to the MRI magnetic field. By using the device of the invention it is thus possible to measure pressure while performing MRI, and thus to obtain both pressure and movement measurements simultaneously.

Since optical fibers are generally of very small diameter, of the order of one-tenth of a millimeter or less, they are not suitable for taking intra-vaginal or intra-rectal measurements: it is therefore difficult to control their positioning and to guarantee that they are kept in contact with the walls of the organ on which pressures are to be measured. In order to mitigate this difficulty, a flexible cavity is provided at the ends of the optical fibers: the flexible cavity serves firstly to come into contact with the organ and transmit the pressure measurement to the optical fibers, and secondly to facilitate accurate observation in the MRI images of the region of the anatomy where the pressure measurement is taken.

Preferably, the device is made out of materials that are flexible and deformable, e.g. polymer materials, so as to enable the device to match the shape of a person's vaginal or rectal cavity, rather than causing the shape of the cavity to match the device. This limits deformation of the person's vaginal or rectal cavity due solely to positioning the measuring device in said cavity, which could otherwise create stresses that are associated solely with positioning the device.

Preferably, the flexible reservoir is filled with a fluid or with a gel. The use of a reservoir filled with a fluid or a gel makes it easy in the MRI images to identify the measuring zone and thus to determine accurately the zone where pressure is measured.

Preferably, the measuring device presents a longitudinal direction, and the pressure measuring surface is a substantially plane surface having its normal perpendicular to the longitudinal direction. The shape of the device is adapted to be used as a vaginal or rectal probe, and the measuring surface of the flexible reservoir is positioned laterally, so as to enable pressure to be measured at various different points merely by positioning and/or orienting the device.

Preferably, at least a portion of the optical fiber sensor is mounted in said flexible reservoir or else in contact with a surface of said flexible reservoir. The optical fiber sensor then makes it possible to measure pressure variations directly inside the reservoir.

### BRIEF DESCRIPTION OF THE DRAWINGS

The invention and its advantages can be better understood on reading the following detailed description of a particular embodiment taken as a non-limiting example and shown in the accompanying drawings, in which:

FIGS. 1 and 2 are diagrammatic views of a measuring device of the invention; and

FIG. 3 is a flow chart of an implementation of the method of the invention.

### DETAILED DESCRIPTION OF THE INVENTION

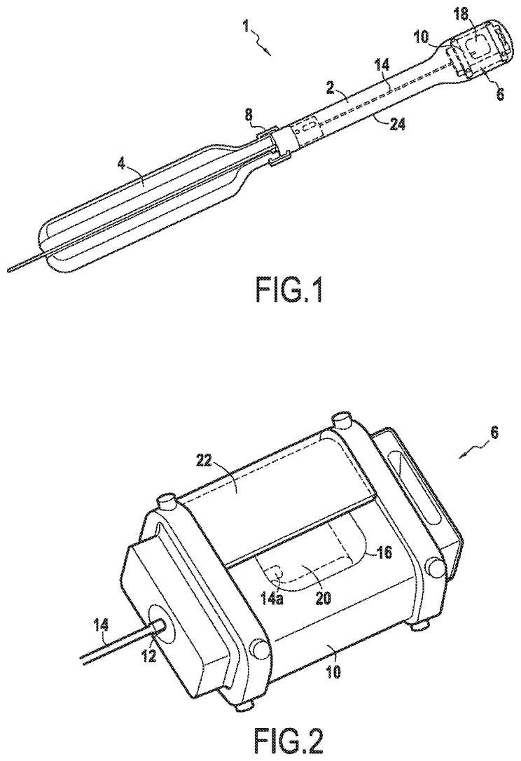

FIG. 1 is a diagram of a device 1 for measuring pressure in an organ of the pelvic cavity.

The measuring device 1 comprises in particular a body 2. The body 2 extends in a longitudinal direction and enables a mechanical connection to be obtained between a positioning handle 4 and pressure measuring means 6. The body 2 is rigid or semi-rigid for the purpose of transmitting mechanical forces exerted on the handle 4, and it is not metallic in order to be compatible with an MRI environment.

The positioning handle 4 is mounted in the longitudinal direction of the body 2 and enables a gynecologist to position and orient the pressure measuring means 6 easily while the device is in use. The positioning handle 4 may in particular be removably mounted, e.g. via a connector 8, at one of the ends of the body 2.

Finally, the device 1 includes the pressure measuring means 6 mounted on the body 2 in the longitudinal direction at its end remote from its end connected to the handle 4.

As shown in FIG. 2, the pressure measuring means 6 comprise a rigid non-metallic housing 10 defining an inside volume that is to receive a fluid or a liquid.

The non-metallic housing 10 also presents a through opening 12 in the longitudinal direction of the device 1 for inserting one or more optical fibers 14 including an end 14a that constitutes an optical fiber sensor that is positioned in the inside volume defined by the non-metallic housing 10. The non-metallic housing 10 also has a lateral opening 16, with its normal being substantially perpendicular to the longitudinal direction of the device 1 and serving to define the outline of a measuring surface 18.

By way of example, the optical fiber sensor 14a may operate by interferometry: an incident light wave is reflected by a dielectric mirror and constitutes a reference wave. The incident beam is also reflected by a diaphragm, i.e. a membrane that is deformable under the effect of an external pressure, and it interferes with the reference beam. The path-length wave difference between the reference beam and the beam reflected by the diaphragm then makes it possible to determine the deformation of the diaphragm, and indirectly to determine the pressure exerted thereon.

The inside volume defined by the non-metallic housing 10 is filled with a fluid or a gel 20 and the lateral opening 16 is covered by a flexible membrane 22 that forms, in the lateral opening 16, the measuring surface 18 of the pressure measuring means 6. The membrane 22 then has the function of deforming in order to transmit pressure to the optical fiber(s) via the fluid or gel present in the cavity, while guaranteeing that the inside volume is sealed. The fluid or the gel provided inside the housing 10 is substantially incompressible, so as to transmit the pressure variations applied to the measuring surface 18 to the end(s) 14a of the optical fiber(s) 14. The quantity of fluid in the inside volume of the non-metallic housing is constant and does not vary. The membrane 22 may be flexible and elastic, or it may be flexible and non-elastic. It thus becomes possible to measure along the axis of the longitudinal direction of the optical fiber(s) 14, i.e. in the longitudinal direction of the device 1, any variation in pressure that is exerted in a direction perpendicular to said longitudinal direction.

Specifically, the optical fibers serve to measure pressure at their distal ends 14a, and they cannot be bent given their brittle nature. The fluid or gel that is in contact both with the measuring surface 18 positioned on a lateral side of the measuring device 1 and with the end(s) of the optical fiber(s) 14 serves to transmit the pressure from the measuring surface 18 to the sensitive surface(s) of the optical fiber(s) 14. There is thus no need for the optical fiber(s) 14 to be curved, which might break them.

Furthermore, the presence of fluid or gel inside the housing 10 also makes it easy to identify and locate the measuring means 6 in MRI images. This enables the local pressure field measured by the device 1 to be characterized accurately.

The measuring means 6 may present the following characteristics: sensitivity of 0.2 millimeters of mercury (mmHg), an optical fiber having a length of 10 meters (m) in order to connect the measuring means 6 to the data acquisition computer, a size less than or equal to 15 millimeters (mm), and a data acquisition frequency greater than or equal to 10 hertz (Hz).

The measuring means 6 are thus completely compatible with an MRI environment. Specifically, firstly the signals transmitted by the optical fiber are not disturbed in any way by the magnetic field or by the radiofrequency waves generated by the MRI during conventional observation sequences of pelvic pathologies, and secondly the presence of the measuring means 6 does not give rise to any artifacts in the images that need to be observed for diagnostic purposes and for making measurements associated with movements.

The device 1 shown in FIGS. 1 and 2 has only one measuring surface 18. Nevertheless, it is also possible to envisage providing a measuring device with a plurality of pressure measuring means 6 arranged along the longitudinal direction of the body, or indeed a housing 10 with a plurality of measuring surfaces 18 arranged around the periphery of the housing 10 so as to provide a device with a plurality of measuring zones. Under such circumstances, each measuring surface 18 should be associated with a respective reservoir of fluid or gel and with one or more optical fibers, and the device can then be used to acquire a plurality of pressure values simultaneously.

The non-metallic housing 10 may be made of hard plastic, e.g. of acrylonitrile butadiene styrene (ABS). The optical fiber(s) is/are then inserted into the non-metallic housing 10. A flexible membrane 22, e.g. made of silicone, is positioned to close the inside volume of the non-metallic housing 10, and the housing is then filled with an aqueous echographic gel by means of a syringe.

In order to limit discomfort for the patient while the device is in use and in order to ensure that it is sealed, the body 2 and the pressure measuring means 6 may in particular be covered in a flexible membrane 24, e.g. made of silicone.

Furthermore, in order to enable suitable measurements to be made of intra-vaginal or intra-rectal pressure, the measuring device 1 is designed to have a shape that guarantees contact between the measuring surface 18 of the measuring means 6 with the wall of the cavity, and also low stress against said cavity. This avoids excessively deforming the cavity, which could modify how the results should be interpreted.

A device 1 is thus obtained that can easily be observed in an MRI environment, and that provides measurements that are not disturbed by said MRI environment.

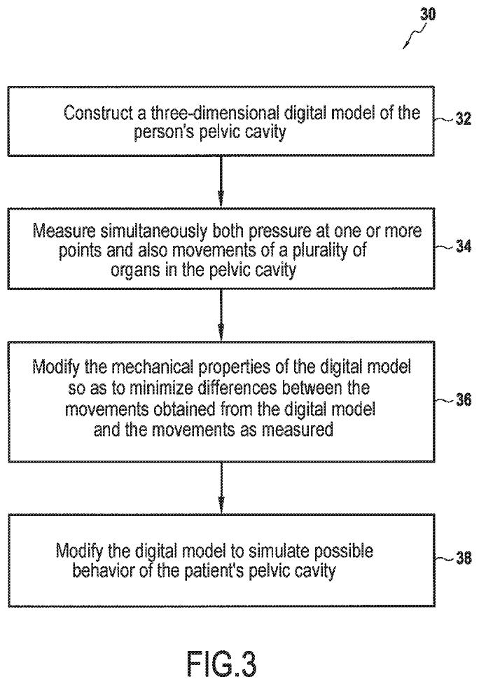

FIG. 3 shows the various steps of the method 30 of determining mechanical properties of a person's pelvic cavity, in particular in non-destructive and in in vivo manner. In a first step 32, a three-dimensional digital model is constructed of the person's pelvic cavity, e.g. using images obtained by static MRI. The digital model may also be made by being subdivided into finite elements in order to make possible the resetting as described below.

In a step 34, measurements are performed simultaneously both of pressure at a plurality of points on the surface of one of the organs and also of movements of a plurality of organs. Pressure measurement may be performed with a device 1 as described with reference to FIGS. 1 and 2, while the movements of organs may be measured by dynamic MRI imaging.

Finally, in a step 36, the mechanical properties of the digital model constructed in step 32 are modified so that these movements obtained by the digital model correspond to the movements measured during the step 34. Such a modification of the digital model may be performed in particular by simulation, using a digital model subdivided into finite elements, simulating the movements obtained for a given pressure field, and by comparing them with the movements as measured during the step 34: the finite element digital model is then reset in order to minimize differences between the two types of movement values.

By means of this method, it is thus possible to obtain a digital model of the patient that combines firstly the three-dimensional shape of the patient and secondly mechanical properties that are specific to the patient.

A last step 38 may then be performed using the resulting digital model. During the step 38, the digital model is modified either in terms of its three-dimensional shape or in terms of its mechanical properties, so as to simulate possible behavior of the patient's pelvic cavity.

Such a step can thus serve to improve diagnosis of pelvic pathologies, e.g. by identifying pathological zones having mechanical properties that are abnormally low or abnormally high, as applies for a prolapse, endometriosis, or a tumor. Likewise, it is also possible to improve therapy of pelvic pathologies by proposing strategies that are better adapted and by making it possible to take account of the specific features of each patient, such as for example simulating various surgical operations and proposing the operation the patient finds most appropriate, or indeed tailoring prostheses to have shapes and mechanical properties that are specifically adapted to the patient. Finally, it is also possible in preventative manner to determine features specific to a woman several months before childbirth and thus to determine complications better and in the much longer term.

Thus, by means of a local measurement of pressure and an overall measurement of movements, which measurements are performed simultaneously, it becomes possible to construct a digital model of a patient's pelvic cavity, which model is representative and reliable. Thereafter, such a model presents the advantage of being able to identify or simulate various abnormalities or complications that might arise with the patient, in order to adapt the procedures or operations that are to be undertaken.

1. A method of determining mechanical properties of the pelvic cavity of a person or an animal, the pelvic cavity including a plurality of organs and the method comprising a step during which pressure is measured at one or more points of the surface of one of the organs of said pelvic cavity, and during which, simultaneously, movements of a plurality of organs of said pelvic cavity are also measured.

2. A method according to claim 1, wherein said organ on the surface of which pressure is measured is the vagina or the rectum.

3. A method according to claim 1, wherein the movements of said pelvic cavity are measured from data obtained by MRI of the person or of the animal.

4. A method according to claim 1, also including a step of constructing a digital model of the pelvic cavity from imaging data of the shape of the pelvic cavity.

5. A method according to the preceding claim 4, wherein construction of the digital model includes subdividing the digital model into finite elements.

6. A method according to claim 4, wherein the mechanical properties used in the digital model are modified in such a manner that the movements obtained with the digital model of said plurality of organs approach the movements as measured when the pressures at said one or more points of the surface of one of the organs of the digital model are equal to the measured pressures.

7. A method according to claim 4, also including, after modifying the mechanical properties of the digital model, a step of modifying the digital model in order to simulate possible mechanical behavior of the pelvic cavity of the person or of the animal.

8. A measuring device for measuring pressure in an organ of the pelvic cavity, the device comprising an optical fiber pressure sensor mounted in a non-metallic housing, and a closed flexible reservoir mounted in said non-metallic housing and having a surface, in particular a flexible surface, that constitutes a pressure measuring surface, the pressure measuring surface being configured to put into contact with a surface of the organ of the cavity and the flexible reservoir being configured to transmit pressure exerted on the measuring surface to the optical fiber sensor.

9. A measuring device according to claim 8, wherein the flexible reservoir is filled with a fluid or with a gel.

10. A measuring device according to claim 8, presenting a longitudinal direction, and wherein the pressure measuring surface is a substantially plane surface having its normal perpendicular to the longitudinal direction.

11. A device according to claim 8, wherein at least a portion of the optical fiber sensor is mounted in said flexible reservoir or else in contact with a surface of said flexible reservoir.

12. A measuring device according to claim 8, wherein the surface of the closed flexible reservoir is a flexible surface.

13. A method according to claim 3, wherein the movements of said pelvic cavity are measured from data obtained by dynamic MRI of the person or of the animal.

14. A method according to claim 4, wherein the step of constructing a digital model of the pelvic cavity from imaging data of the shape of the pelvic cavity comprises a step of constructing a digital model of the pelvic cavity from data obtained by static MRI of the person or of the animal.

15. A method according to claim 4, wherein the step of constructing a digital model of the pelvic cavity from imaging data of the shape of the pelvic cavity comprises a step of constructing a digital model of the pelvic cavity from data obtained by static MRI of the person or of the animal, and from standard mechanical properties.

16. A method according to claim 7, wherein the step of modifying the digital model comprises a step of modifying the shape of the digital model or modifying a mechanical property.
